(19) United States
(12) Patent Application Publication
(10) Pub. No.: US 2005/0159371 A1
(43) Pub. Date: Jul. 21, 2005

(54) Process for producing erythromycin a derivative

(75) Inventors: Masato Kashimura, Tokyo (JP); Takeshi Kuwada, Tokyo (JP)

Correspondence Address: BURNS DOANE SWECKER & MATHIS L L P, POST OFFICE BOX 1404, ALEXANDRIA, VA, 22313-1404, US

(73) Assignee: Taisho Pharmaceutical Co., Ltd, Toshima-ku, Japan (JP)

(21) Appl. No.: 10/504,088

(22) PCT Filed: Feb. 12, 2003
(86) PCT No.: PCT/JP03/01412

(30) Foreign Application Priority Data
Feb. 13, 2002 (JP) 202-35510

(51) Int. Cl. C07H 17/08; A61K 31/7052

(52) U.S. Cl. 514/29; 536/7.4

(57) ABSTRACT

An efficient process for producing an 11,12-cyclic carbamate 6-O-substituted ketolide derivatives having the natural stereochemistry at the 10-position.

### BACKGROUND OF THE INVENTION

1. Field of the Invention

The present invention relates to a process for producing erythromycin A derivatives and, more particularly, to a process for producing erythromycin A 11,12-cyclic carbamate 6-O-substituted ketolide derivatives.

2. Description of the Related Art

Erythromycin is an excellent antibacterial agent and has been widely used clinically since the 1950's, but it is unstable to acids. In order to overcome this drawback of erythromycin, a large number of erythromycin derivatives have been synthesized, and some of these derivatives are clinically used as excellent antibiotics. For example, clarithromycin (6-O-methylerythromycin A, U.S. Pat. No. 4,331,803) is widely used as a therapeutic agent of respiratory infections due to its excellent biological properties. There have been recently reported the derivatives which are generically called ketolides, and have potent antibacterial activity against macrolide-resistant bacteria (Bioorganic and Medicinal Chemistry Letters, Vol. 9, 3075-3080 (1999), Journal of Medicinal Chemistry, Vol. 43, 1045-1049 (2000), Journal of Antibiotics, Vol. 54, 664-678 (2001)).

These ketolides are structurally characterized in that the cladinose at the 3-position of erythromycin A is removed and converted into a carbonyl group, the 6-hydroxyl group is alkylated, and the 11,12-hydroxyl groups are converted into a cyclic carbamate. It has ever been reported that by reacting a 10,11-anhydro-12-O-imidazolyl carbonyl derivative, in which the 3-position of erythromycin A has been converted into a carbonyl group, with liquid ammonia or aqueous ammonia so as to carry out 11,12-cyclic carbamate formation, the natural R configuration and the unnatural S configuration with respect to the stereochemistry at the 10-position are obtained as a mixture (Journal of Medicinal Chemistry, Vol. 41, 4080-4100 (1998), Journal of Medicinal Chemistry, Vol. 43, 1045-1049 (2000)). It is therefore necessary to carry out cyclic carbamate formation of the 11,12-hydroxyl groups before chemical modification at the 3-position in order to produce a ketolide in high yield. This imposes restrictions on the order of the chemical modifications in the production of the ketolide, resulting in a long and restricted production process.

### SUMMARY OF THE INVENTION

An object of the present invention is to provide a selective and efficient process for producing erythromycin A 11,12-cyclic carbamate derivatives.

As a result of an intensive investigation, the present inventors have found a process for leading to an 11,12-cyclic carbamate 6-O-substituted ketolide derivative having the natural stereochemistry at the 10-position can be obtained selectively and efficiently by subjecting a 10,11-anhydro-12-O-aminocarbonyl derivative, which has been derived from an 11,12-cyclic carbonate derivative of erythromycin A with the 3-position thereof converted into a carbonyl group, to 11,12-cyclic carbamate formation with a specific type of base in combination with an imidazole derivative or an alcohol, thereby the present invention has been accomplished.

In further detail, the process is useful for production of 3-deoxy-3-oxo-6-O-(3-(3-quinolyl)-2-propen-1-yl)-5-O-desosaminylerythronolide A 11,12-cyclic carbamate, which has been reported to have very strong antibacterial activity. In this process, 3-deoxy-3-oxo-6-O-(3-(3-quinolyl)-2-propen-1-yl)-5-O-desosaminylerythronolide A 11,12-cyclic carbonate, which is described in U.S. Pat. No. 5,866,549, is used as a starting material; it is converted by a standard method into a compound in which the 2′-hydroxyl group is protected, a 10,11-anhydro derivative (III) is derived from the 11,12-cyclic carbonate structure using a base such as triethylamine, 1,8-diazabicyclo[5,4,0]undec-7-ene, potassium carbonate, sodium carbonate, barium carbonate, lithium carbonate, sodium hydroxide, potassium hydroxide, lithium hydroxide, barium hydroxide, or sodium hydride, a 12-hydroxyl group is then activated using an activating agent such as N,N′-carbonyldiimidazole, phosgene, phosgene dimer, triphosgene, or ethyl chloroformate and then reacted with liquid ammonia, ammonia gas, or aqueous ammonia to give a 12-O-aminocarbonyl derivative (IV), and the compound (IV) is further converted into the 11,12-cyclic carbamate structure having the natural stereochemistry at the 10-position using a base such as 1,8-diazabicyclo[5,4,0]undec-7-ene, sodium hydroxide, potassium hydroxide, lithium hydroxide, lithium hydroxide hydrate, barium hydroxide, barium hydroxide hydrate, or sodium hydride, and an imidazole derivative (imidazole or methylimidazole) or an alcohol (methanol, ethanol, or isopropanol).

That is, the present invention is the process for producing compound (V) defined below, which comprises the steps of:

(A) protecting the 2′-hydroxyl group of a compound (I) represented by the formula below, which is used as a starting material, 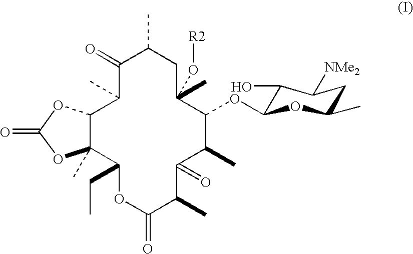
(wherein R2 represents an alkyl group having 1 to 4 carbon atoms, allyl group, and an allyl group substituted with an aromatic or hetero ring having 5 to 12 carbon atoms) with an R1 group (R1 represents acetyl group, benzoyl group, propionyl group, trimethylsilyl group, triethylsilyl group, and t-butyldimethylsilyl group) to give a compound (II) represented by the formula below 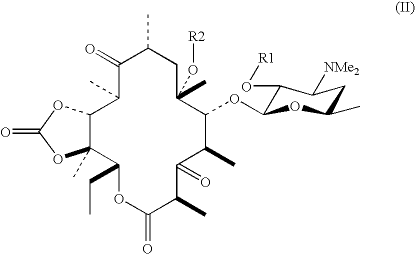
(wherein R1 and R2 denote the same as above);

(B) treating the compound (II) with a base to give a compound (III) represented by the formula below 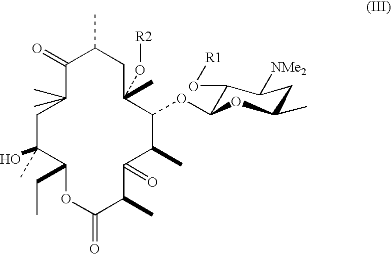
(wherein R1 and R2 denote the same as above);

(C) activating the 12-hydroxyl group of the compound (III) using an activating agent, and then reacting with a compound represented by formula R3-NH₂ (wherein R3 represents hydrogen atom, amino group, an alkyl group having 1 to 4 carbon atoms, and an alkyl group having 1 to 4 carbon atoms that is substituted with a group selected from pyridyl group, quinolyl group, imidazolyl group, and pyridylimidazolyl group) to give a compound (IV) represented by the formula below 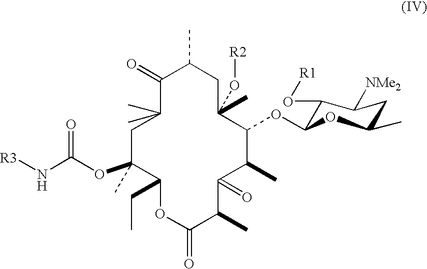
(wherein R1, R2, and R3 denote the same as above); and

(D) subjecting the compound (IV) to cyclic carbamate formation using one or more types of compound selected from the group consisting of 1,8-diazabicyclo[5,4,0]undec-7-ene, cesium carbonate, lithium hydroxide, lithium hydroxide hydrate, potassium hydroxide, sodium hydroxide, barium hydroxide, barium hydroxide hydrate, and sodium hydride in combination with one or more types of compound selected from the group consisting of imidazole, methylimidazole, methanol, ethanol, and isopropanol to give a compound (V) represented by a formula below 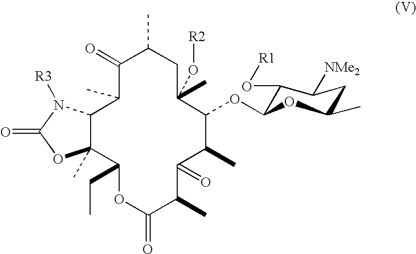
(wherein R1, R2, and R3 denote the same as above).

The alkyl group having 1 to 4 carbon atoms referred to in the present invention includes a linear or branched alkyl group, and examples thereof include methyl group, ethyl group, propyl group, isopropyl group, butyl group, and t-butyl group. The base includes triethylamine, 1,8-diazabicyclo[5,4,0]undec-7-ene, potassium carbonate, sodium carbonate, barium carbonate, lithium carbonate, sodium hydroxide, potassium hydroxide, lithium hydroxide, lithium hydroxide hydrate, barium hydroxide, barium hydroxide hydrate, and sodium hydride. The activating agent includes N,N′-carbonyldiimidazole, phosgene, phosgene dimer, triphosgene, and ethyl chloroformate. The compound represented by formula R3-NH₂ includes ammonia, hydrazine, an alkylamine having 1 to 4 carbon atoms, and an alkylamine having 1 to 4 carbon atoms that is substituted with a group selected from the group consisting of pyridyl group, quinolyl group, imidazolyl group, and pyridylimidazolyl group. The alkylamine having 1 to 4 carbon atoms includes methylamine, ethylamine, propylamine, butylamine, and isopropylamine.

The present invention relates to a production process illustrated in the reaction scheme below. It relates to a process for producing compound (V) using compound (I) as a starting material, which is described in U.S. Pat. No. 5,866,549. 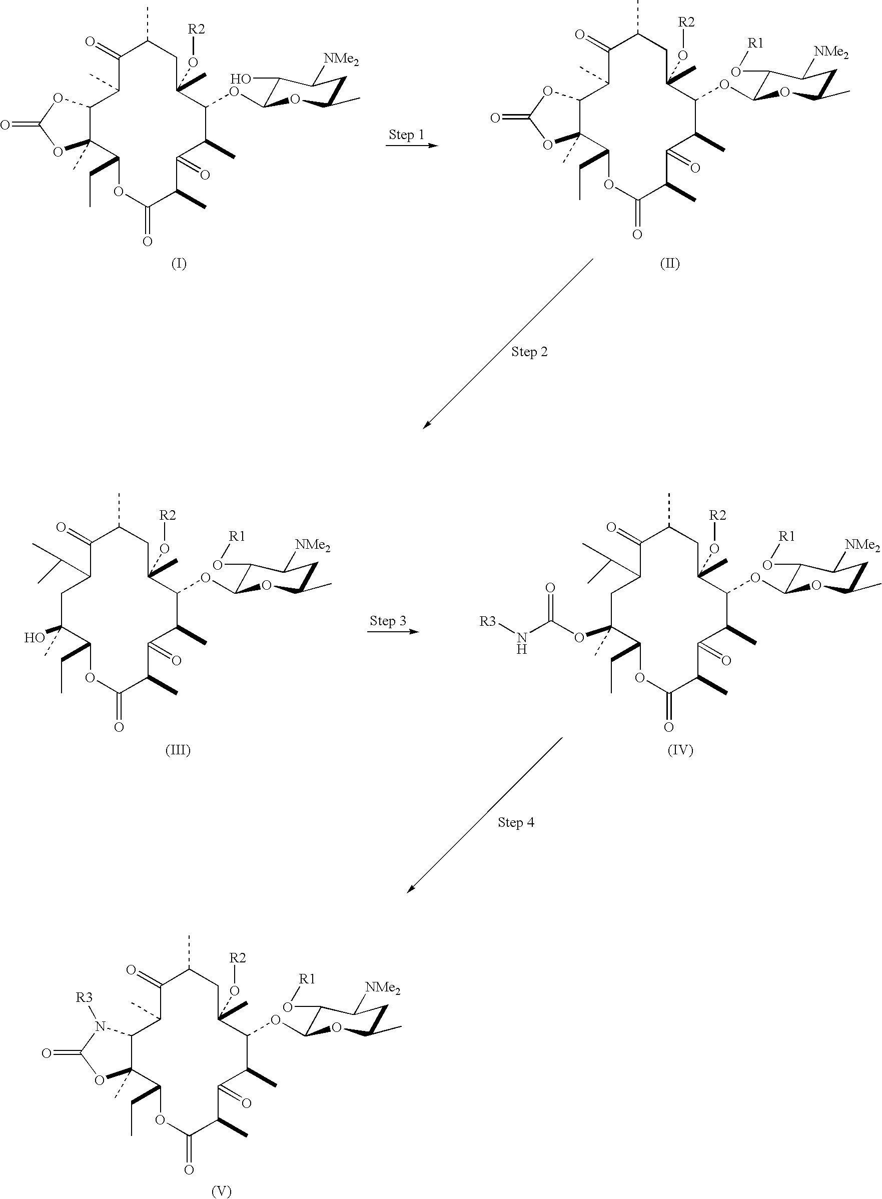
(wherein R1, R2, and R3 denote the same as above.)

Step 1: Compound (I), which is described in U.S. Pat. No. 5,866,549, is used as a starting material, and the 2′-hydroxyl group thereof is protected with an R1 group (R1 denotes the same as above) by a standard method to give a compound (II).

Step 2: The compound (II) is treated with a base in an inert solvent at a temperature from 0° C. to the boiling temperature of the solvent, and preferably at a temperature from room temperature to the boiling temperature of the solvent, to give a compound (III). The inert solvent available includes toluene, tetrahydrofuran, acetone, ethyl acetate, isopropyl acetate, methylene chloride, and the base available includes triethylamine, 1,8-diazabicyclo[5,4,0]undec-7-ene, potassium carbonate, sodium carbonate, barium carbonate, lithium carbonate, sodium hydroxide, potassium hydroxide, lithium hydroxide, barium hydroxide, sodium hydride.

Step 3: The 12-hydroxyl group of the compound (III) obtained in Step 2 is activated with an activating agent in an inert solvent at a temperature between −10° C. and 60° C., and preferably 0° C. to room temperature, and then reacted at the same reaction temperature with a compound represented by the formula R3-NH₂ (whrein R3 denotes the same as above) to give a compound (IV). The inert solvent referred to here is the same as that used in Step 2, and the activating agent available includes N,N′-carbonyldiimidazole, phosgene, phosgene dimer, triphosgene, ethyl chloroformate. The compound represented by the formula R3-NH₂ includes ammonia, hydrazine, an alkylamine having 1 to 4 carbon atoms, and an alkylamine having 1 to 4 carbon atoms that is substituted with a group selected from pyridyl group, quinolyl group, imidazolyl group, pyridylimidazolyl group, preferably ammonia, hydrazine, and an alkylamine having 1 to 4 carbon atoms.

Step 4: The compound (IV) obtained in Step 3 is subjected to cyclic carbamate formation in an inert solvent at a temperature between −10° C. and 60° C., and preferably 0° C. to room temperature, using a specific type of base in combination with an imidazole derivative or an alcohol to give the target compound (V). The inert solvent referred to here is the same as that used in Step 2; the base available includes one or more types selected from 1,8-diazabicyclo[5,4,0]undec-7-ene, cesium carbonate, lithium hydroxide, lithium hydroxide hydrate, potassium hydroxide, sodium hydroxide, barium hydroxide, barium hydroxide hydrate, and sodium hydride. The imidazole derivative available includes imidazole and methylimidazole. The alcohol available includes methanol, ethanol, and isopropanol. The imidazole derivative and the alcohol may be used singly or in a combination of two or more types. By making the alcohol serve also as the solvent, 11,12-cyclic carbamate formation and removal of the protecting group at the 2′-position can be carried out at the same time, and the 2′-hydroxyl derivatives and the 2′-protected derivatives can be selectively produced freely as required.

### PREFERRED EMBODIMENT OF THE INVENTION

The present invention is explained below in further detail.

### EXAMPLE 1

### Production of 10,11-anhydro-2′-O-benzoyl-3,11-dideoxy-3-oxo-6-O-(3-(3-quinolyl)-2-propen-1-yl)-5-O-desosaminylerythronolide A

A compound (20.0 g) obtained by subjecting 3-deoxy-3-oxo-6-O-(3-(3-quinolyl)-2-propen-1-yl)-5-O-desosaminylerythronolide A 11,12-cyclic carbonate, which is described in Example 75 of U.S. Pat. No. 5,866,549, to 2′-O-benzoylation by a standard method was dissolved in tetrahydrofuran (400 mL), anhydrous potassium carbonate (15.9 g, 5 equivalents) was added thereto, and the mixture was heated and refluxed for 23 hours. After allowing it to cool, a precipitate was filtered off (washed with ethyl acetate (200 mL)), the filtrate thus obtained was washed with saturated brine, dried with anhydrous magnesium sulfate, and filtered, and the solvent was distilled off under vacuum. The crude product thus obtained was subjected to purification by silica gel column chromatography (eluent acetone:hexane:triethylamine=3:10:0.2 to 5:10:0.2) to give the title compound (19.8 g).

¹H NMR(500 MHz, CDCl₃) δ (ppm): 2.03(s, 3H, 10-Me), 4.97(dd, 1H, J=10.3, 2.6 Hz, 13-H), 5.07(dd, 1H, J=10.4, 7.6 Hz, 2′-H), 6.48(s, 1H, 11-H), 8.89(d, 1H, J=2.1 Hz, 2-H of quinoline)

¹³C NMR(125 MHz, CDCl₃) δ (ppm): 73.5(12-C), 139.8(10-C), 141.1(11-C), 208.3(3-C & 9-C)

ESI-MS: m/z=849.2[M+Na]⁺

### EXAMPLE 2

### Production of 10,11-anhydro-12-O-aminocarbonyl-2′-O-benzoyl-3,11-dideoxy-3-oxo-6-O-(3-(3-quinolyl)-2-propen-1-yl)-5-O-desosaminylerythronolide A

The compound (18.6 g) obtained in Example 1 was dissolved in tetrahydrofuran (372 mL), carbonyldiimidazole (10.9 g, 3 equivalents) and 1,8-diazabicyclo[5,4,0]undec-7-ene (342 mg, 0.1 equivalents) were added thereto, and the mixture was stirred for 3 hours while cooling. Subsequently, ammonia gas was passed through the mixture for 18.5 hours while ice cooling. Toluene (400 mL) and saturated brine (100 mL) were added to the mixture at room temperature and separated, the organic phase thus obtained was washed twice with saturated brine (100 mL), dried with anhydrous magnesium sulfate, and filtered, and the solvent was then distilled off under vacuum to give the title compound (20.2 g).

¹H NMR(500 MHz, CDCl₃) δ (ppm): 1.90(s, 3H, 10-Me), 5.82(m, 1H, 13-H), 6.75(s, 1H, 11-H)

¹³C NMR(125 MHz, CDCl₃) δ (ppm): 138.3(10-C), 141.1(11-C), 154.4(12-O—CO—NH₂)

ESI-MS: m/z=870.3[M+H]⁺

### EXAMPLE 3

### Production of 2′-O-benzoyl-3-deoxy-3-oxo-6-O-(3-(3-quinolyl)-2-propen-1-yl)-5-O-desosaminylerythronolide A 11,12-cyclic carbamate

The compound (15.0 g) obtained in Example 2 was dissolved in toluene (500 mL), and the solvent was then distilled off under vacuum. The residue thus obtained was dissolved in anhydrous toluene (150 mL), imidazole (2.35 g, 2 equivalents) and cesium carbonate (5.62 g, 1 equivalent) were added thereto, and the mixture was stirred at room temperature for 3.5 hours. Saturated aqueous ammonium chloride (250 mL) was added to the reaction mixture, the mixture was separated, and then, the aqueous phase was extracted twice with toluene (50 mL). The combined organic phases were washed three times with saturated aqueous ammonium chloride (50 mL), washed with saturated brine (50 mL), dried with anhydrous magnesium sulfate, and filtered, and the solvent was distilled off under vacuum to give the title compound (14.0 g, yield 93.3%).

¹H NMR(500 MHz, CDCl₃) δ (ppm): 2.26(s, 6H, NMe₂), 2.88(m, 1H, 10-H), 3.86(s, 1H, 11-H), 5.49(s, 1H, NH)

¹³C NMR(125 MHz, CDCl₃) δ (ppm): 37.2(10-C), 58.1(11-C), 83.4(12-C), 157.6(12-O—CO-1-11)

ESI-MS: m/z=892.4[M+Na]⁺

### EXAMPLE 4

The compound (500 mg) obtained in Example 2 was subjected to a reaction in the same manner as in Example 3 using cesium carbonate (56 mg, 0.3 equivalents) and imidazole (19.5 mg, 0.5 equivalents) in anhydrous toluene (5 mL) to give the same compound (479 mg, yield 95.8%) as that obtained in Example 3.

### EXAMPLE 5

The compound (500 mg) obtained in Example 2 was subjected to a reaction in the same manner as in Example 3 using cesium carbonate (188 mg, 1 equivalent) and methanol (37 mg, 2 equivalents) in anhydrous toluene (5 mL) to give the same compound (481 mg, yield 96.2%) as that obtained in Example 3.

### EXAMPLE 6

The compound (500 mg) obtained in Example 2 was subjected to a reaction in the same manner as in Example 3 using potassium hydroxide (38 mg, 1 equivalent) and imidazole (78 mg, 2 equivalents) in anhydrous toluene (5 mL) to give the same compound (425 mg, yield 85.0%) as that obtained in Example 3.

### EXAMPLE 7

The compound (500 mg) obtained in Example 2 was subjected to a reaction in the same manner as in Example 3 using anhydrous lithium hydroxide (14 mg, 1 equivalent) and imidazole (78 mg, 2 equivalents) in anhydrous toluene (5 mL) to give the same compound (464 mg, yield 92.8%) as that obtained in Example 3.

### EXAMPLE 8

The compound (500 mg) obtained in Example 2 was subjected to a reaction in the same manner as in Example 3 using 1,8-diazabicyclo[5,4,0]undec-7-ene (87.5 mg, 1 equivalent) and imidazole (78 mg, 2 equivalents) in anhydrous toluene (5 mL) to give the same compound (481 mg, yield 96.2%) as that obtained in Example 3.

### EXAMPLE 9

### Production of 3-deoxy-3-oxo-6-O-(3-(3-quinolyl)-2-propen-1-yl)-5-O-desosaminylerythronolide A 11,12-cyclic carbamate (compound described in Example 104 of U.S. Pat. No. 5,866,549)

3-Deoxy-3-oxo-6-O-(3-(3-quinolyl)-2-propen-1-yl)-5-O-desosaminylerythronolide A 11,12-cyclic carbonate, which is described in Example 75 of U.S. Pat. No. 5,866,549, was subjected to 2′-O-acetylation by a standard method and then to reactions in the same manner as in Examples 1 and 2 to give a compound (501 mg) to which was added 1,8-diazabicyclo[5,4,0]undec-7-ene (9 mg, 0.1 equivalents), and the mixture was stirred in methanol (10 mL) at room temperature for 44 hours. The solvent was distilled off under vacuum, to the residue thus obtained was added toluene (100 mL), and it was washed twice with saturated brine (10 mL). The washings were subjected to extraction using ethyl acetate (10 mL), the combined organic phases were dried with anhydrous magnesium sulfate and filtered, and the solvent was distilled off under vacuum. The residue thus obtained was subjected to purification by silica gel column chromatography to give the title compound (379 mg, yield 79.8%).

### EXAMPLES 10-13

The compound obtained in Example 2 was reacted in anhydrous toluene under the conditions shown in Table 1 to give the same compound as that obtained in Example 3 in the yields shown in the table.

TABLE 1

| | | | Tem- | | HPLC |
|---|---|---|---|---|---|
| Example | Reagent | | perature | Time | (Pa %:254 nm) |
| 10 | Sodium hydride | 1eq | r.t. | 5 h | 80.1 |
| | Imidazole | 2eq | | | |
| 11 | Sodium hydroxide | 1eq | r.t. | 5 h | 81.6 |
| | Imidazole | 2eq | | | |
| 12 | Lithium hydroxide | 1eq | r.t. | 13 h | 85.3 |
| | monohydrate | | | | |
| | Imidazole | 2eq | | | |
| 13 | Barium hydroxide | 1eq | r.t. | 18 h | 86.6 |
| | octahydrate | | | | |
| | Imidazole | 2eq | | | |

1. A process for producing a compound (V) comprising:
(A) protecting the 2′-hydroxyl group of a compound (I) represented by a formula below, which is used as a starting material, 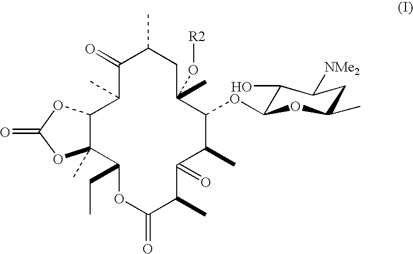
(wherein R2 represents an alkyl group having 1 to 4 carbon atoms, allyl group, and an allyl group substituted with an aromatic or hetero ring having 5 to 12 carbon atoms) with an R1 group (R1 represents acetyl group, benzoyl group, propionyl group, trimethylsilyl group, triethylsilyl group, and t-butyldimethylsilyl group) to give a compound (II) represented by a formula below 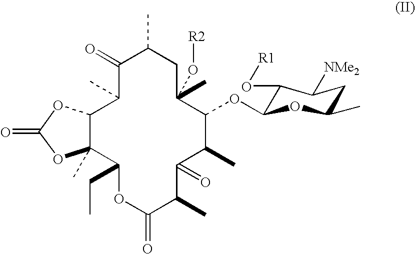
(wherein R1 and R2 denote the same as above);
(B) treating said compound (II) with a base to give a compound (III) represented by a formula below 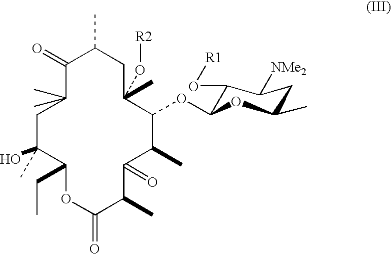
(wherein R1 and R2 denote the same as above);
(C) activating the 12-hydroxyl group of said compound (III) using an activating agent, and then reacting with a compound represented by formula R3-NH₂ (wherein R3 represents hydrogen atom, amino group, an alkyl group having 1 to 4 carbon atoms, and an alkyl group having 1 to 4 carbon atoms that is substituted with a group selected from pyridyl group, quinolyl group, imidazolyl group, and pyridylimidazolyl group) to give a compound (IV) represented by a formula below 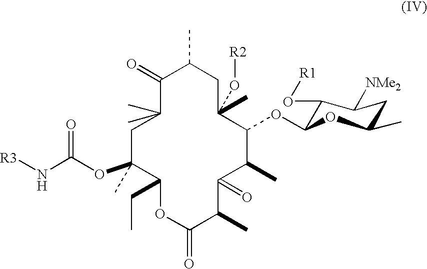
(wherein R1, R2, and R3 denote the same as above); and
(D) subjecting said compound (IV) to cyclic carbamate formation using one or more types of compound selected from a group consisting of 1,8-diazabicyclo[5,4,0]undec-7-ene, cesium carbonate, lithium hydroxide, lithium hydroxide hydrate, potassium hydroxide, sodium hydroxide, barium hydroxide, barium hydroxide hydrate, and sodium hydride in combination with one or more types of compound selected from a group consisting of imidazole, methylimidazole, methanol, ethanol, and isopropanol to give said compound (V) represented by a formula below 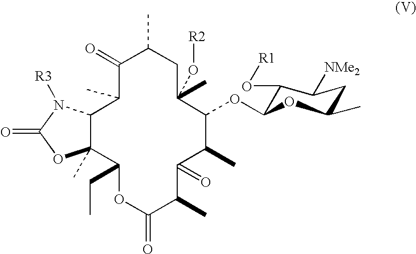
(wherein R1, R2, and R3 denote the same as above).
